(19) United States
(12) Patent Application Publication
(10) Pub. No.: US 2014/0017778 A1
(43) Pub. Date: Jan. 16, 2014

(54) SOLAR PHOTOBIOREACTOR WITH CONTROLLED VOLUME FLOW DILUTION

(71) Applicants: Jean-François Cornet, Durtol (FR); Vincent Goetz, Pollestres (FR); Gaël Plantard, Narbonne (FR); Roger Garcia, Cabestany (FR); Pascal Lafon, Aydat (FR); Frédéric Joyard, Saint Saturnin (FR)

(72) Inventors: Jean-François Cornet, Durtol (FR); Vincent Goetz, Pollestres (FR); Gaël Plantard, Narbonne (FR); Roger Garcia, Cabestany (FR); Pascal Lafon, Aydat (FR); Frédéric Joyard, Saint Saturnin (FR)

(21) Appl. No.: 13/989,406

(22) PCT Filed: Nov. 25, 2011
(86) PCT No.: PCT/EP2011/071002

§ 371 Date: Oct. 8, 2013

(30) Foreign Application Priority Data
Nov. 25, 2010 (FR) 1059761

(51) Int. Cl. C12M 1/00

(52) U.S. Cl. CPC C12M 21/02
USPC 435/292.1

(57) ABSTRACT

The invention relates to a sunlight-guiding module (2) for a photobioreactor (1) comprising a reaction chamber (11), the module (2) comprising: —a concentrating device (21) placed outside the reaction chamber (11) in order to concentrate the sunlight into a concentrated beam; —a scattering device (24) placed in the reaction chamber (11); characterised in that the module (2) also comprises a homogenising device (22) placed between the concentrating device (21) and the scattering device (24) in order to make the concentrated beam homogeneous before the concentrated beam enters the scattering device (24). The invention also relates to a light-guiding panel (3) comprising at least two light-guiding modules (2). Finally, the invention relates to a photoreactor (1) comprising a reaction chamber (11), characterised in that it also comprises a light-guiding module (2) or panel (3).

### FIELD OF THE INVENTION

The invention concerns the general field of photoreactors. More particularly, the invention concerns the field of photobioreactors and modules for capturing and distributing sunlight for photobioreactors used for producing photosynthetic microorganisms.

### TECHNOLOGICAL BACKGROUND

Today, two techniques are used for producing photosynthetic microorganisms: open-air techniques (for example open pools) and closed-reactor techniques

Closed-reactor techniques have the advantage of allowing for:
keeping the culture of photosynthetic microorganisms sterile, thus preventing at least contaminations;
strict control of water and nutriment supply;
control of carbon dioxide transfer and jointly the pH, increasing the surface productivity;
culture at high biomass concentrations, also increasing productivity but also optimising compactness, this resulting in a reduction in the energy costs associated with the circulation of fluids and the thermal control of the reactor.

Closed-reactor techniques are generally implemented by photobioreactors with direct capture of sunlight through a transparent wall.

However, this situation in which the density of incident direct solar flow prevails leads to functioning with very low thermodynamic efficiency of conversion of photosynthesis. These techniques therefore require high surface availability.

It is also known that using photobioreactors for which the sunlight is first captured and then brought inside the photobioreactors in order to be distributed therein at low flux density may significantly increase the thermodynamic efficiency of the photosynthesis, as in the document WO 2007/134141. This increases accordingly the surface productivity of the photobioreactors. The capture can be achieved in various ways and makes it possible to form a beam concentrating the sunlight. This beam is then sent to a light guide in order to bring the light flux into the reaction chamber of the photobioreactor and be distributed therein.

However, in such a photobioreactor, the guide or scattering device does not make it possible to diffuse the light homogeneously inside the photobioreactor, thus reducing the efficiency of the bioreactor. Equally, the guide or scattering device may suffer deterioration due to an excessively high temperature rise within them, sometimes making them melt. Finally, the presence of numerous structures diffusing the light inside the reaction chamber may give rise to problems of cell adhesion or poor mixing leading to a loss of efficiency.

### SUMMARY

One of the aims of the invention is to overcome at least one drawback of the prior art.

In particular, one aim of the invention is to afford better efficiency of the direct capture of the sun by providing a module for capturing and distributing sunlight for a photoreactor comprising a reaction chamber, the module comprising:
a concentrator placed outside the reaction chamber in order to concentrate the sunlight into a concentrated beam;
a scattering device to be placed in the reaction chamber in order to introduce the concentrated beam into the reaction chamber and diffuse the concentrated beam inside the reaction chamber;

characterised in that the module also comprises a homogeniser placed between the concentrator and the scattering device, outside the reaction chamber, in order to make the concentrated beam homogeneous before the concentrated beam enters the scattering device.

This module has the advantage of enabling homogenisation of the beam concentrated by the concentrator on the scattering device. Indeed, it has been discovered that light diffusion inhomogeneity inside the photoreactor was partly due to inhomogeneity of the illumination of the scattering device by the concentrated beam. Indeed, a concentrated beam has a Gaussian-shaped flux density profile. Thus the centre of the scattering device receives too much light whereas its periphery does not receive sufficient light. The centre of the scattering device may then heat excessively and melt.

Other optional and non-limitative features are:
the module also comprises a light guide for guiding the light from the concentrator to the scattering device, the light guide being placed between the homogeniser and the scattering device outside the reaction chamber;
the module also comprises a near-infrared filter for filtering the sunlight so that the sunlight is free from near-infrared radiation;
the concentrator is a Fresnel-lens concentrator comprising a Fresnel lens having a focus;
the module may comprise a compound-mirror collector with an almost punctiform focus placed between the Fresnel-lens concentrator and the homogeniser and in front of the focus of the Fresnel lens to redirect the concentrated beam;
the concentrator is a concentrator of the Cassegrain type;
the light guide and the scattering device form a single element composed of optical fibres;

each optical fibre having a diffusing part, the diffusing parts of the optical fibres form the scattering device; and

each optical fibre having a guide part, the guide parts of the optical fibres are collected together into a strand forming the light guide.

The invention also provides a panel for capturing and distributing sunlight, comprising at least two modules as described above, comprising a concentrator with a Fresnel lens.

The invention also provides a photoreactor, in particular a photobioreactor, comprising a reaction chamber, characterised in that it also comprises a module or panel as described above.

One advantage with such a photoreactor is related to the use of the light-guide module. The advantages of the module are therefore afforded to such a photoreactor. Additionally, thanks to the possibility of grouping together the modules into a panel, the photoreactor may have various dimensions, the number of modules used having simply to be adapted.

Other optional and non-limitative features of this photoreactor are:
the diffusing parts of the optical fibres of the module or panel being arranged tensioned and parallel to each other inside the reaction chamber in a triangular meshing;
the photoreactor also comprises at least three gratings disposed parallel one above another, each being formed by a frame and wires tensioned inside the frame parallel to one another in one direction, and the direction of the wires of a grating forming, with the direction of the wires of a grating above, an angle of ±60° to keep the diffusing parts of the optical fibres in a triangular mesh; and
the photoreactor also comprises a gas trap with an external recirculation loop and a central return for mixing effectively the content of the reaction chamber.

### PRESENTATION OF THE DRAWINGS

Other features, objectives and advantages will become apparent from the following detailed description with reference to the illustrative and non-limiting drawings, among which:

FIG. 1 is a schematic view of an example of a photobioreactor of the invention;

FIG. 2 is a perspective view of a first embodiment of a light-guide module with a concentrator with Fresnel lens for a photobioreactor;

FIG. 3 is a section view of the module of FIG. 1;

FIG. 4 is a perspective view of a panel composed of several modules of FIG. 1;

FIG. 5 is a perspective view of an assembly consisting of a photobioreactor and a panel of the same type as the panel in FIG. 4;

FIG. 6 is a perspective view of a photobioreactor and light-guide module assembly according to a second embodiment with a Cassegrain concentrator;

FIG. 7 is a partial perspective view of an embodiment of a homogeniser used in the modules shown in FIGS. 1 to 6;

FIG. 8 is a close-up view of a group of optical fibres in strands as used with the modules in FIGS. 1 to 6;

FIG. 9 is a perspective view of a grating to be placed inside the reaction chamber of a photobioreactor for parallel distribution of the optical fibres into a triangular mesh;

FIG. 10 is a view of the inside of the reaction chamber in which five gratings of FIG. 9 are used for distributing the optical fibres;

FIG. 11 is a perspective view, partially cut-away, of a sensor used in the modules in FIGS. 1 to 5;

FIG. 12 is a perspective view of an example of a sun tracker used with the modules of FIGS. 1 to 6;

FIG. 13 is a cross section view of a reaction chamber between two gratings and showing the distribution of optical fibres in a triangular mesh inside the reaction chamber.

### DETAILED DESCRIPTION

### Module for Capturing and Distributing Sunlight

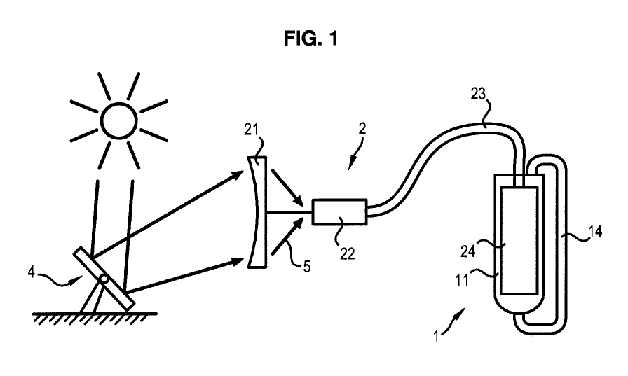

With reference to FIGS. 1 to 6 and 10, a module for capturing and distributing sunlight for a photobioreactor is described below.

The role of the module 2 is to capture a light flux coming from sunlight and to transport it inside a photobioreactor 1 comprising a reaction chamber 11 and distributing the captured light flux as homogeneously as possible inside the reaction chamber 11.

The reaction chamber 11 is an internal space of the photobioreactor 1 in which the microorganisms in culture in a solution are placed. The microorganisms receive therein the light flux and the nutriments necessary for their growth and development.

The module 2 comprises a concentrator 21 for concentrating the light flux coming from the sunlight into a concentrated beam 5. The concentrator 21 may have several embodiments that will be described below.

The module 2 also comprises a scattering device 24 to be placed inside the reaction chamber 11 in order to diffuse therein the light flux of the concentrated beam 5.

The module 2 also comprises a light guide 23, to be placed outside the reaction chamber 11, in order to conduct the light flux of the concentrated beam 5 from the concentrator 21 to the scattering device 24.

The light flux of the concentrated beam 5 may be very high in an area close to the centre of the concentrated beam 5. In the majority of cases, the light flux of the concentrated beam 5 has a Gaussian profile.

When the concentrated beam 5 enters the light guide 23, this area with a high light flux may damage or even destroy the light guide 23 and also the scattering device 24. In the case of optical fibres 25 fulfilling the role of light guide 23 and scattering device 24, as described hereinafter, the area with a high light flux may melt the optical fibres 25.

In order to prevent damage to the light guide 23 and/or the scattering device 24, the module 2 also comprises a homogeniser 22 for homogenising the light flux of the concentrated beam 5 on the light guide 23.

The homogeniser 22 makes the light flux of the concentrated beam 5 uniform by flattening its profile. Because of this the risk of overheating in the area of high light flux of the concentrated beam 5 is also avoided.

The homogeniser 22 may be implemented in the form of a fibre rod. The profile of the flux density of the light in the concentrated beam 5 is made uniform by multiple almost total reflections made by the rays of the concentrated beam 5 inside the fibre rod 22 before emerging therefrom. Indeed, the rays of the concentrated beam 5 enter the optical array 22 at different angles and follow different optical paths inside the optical array 23.

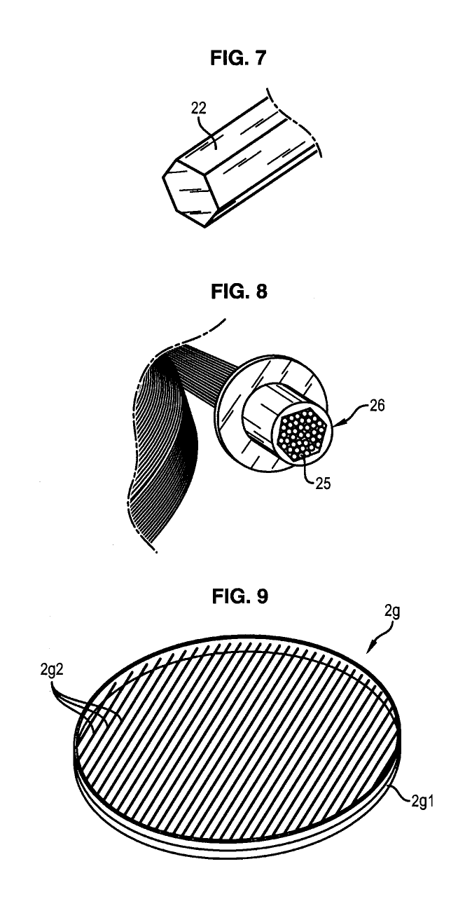

The length and form of the fibre rod 22 may vary. For example, the fibre rod 22 is parallelepipedal or pyramidal in shape. The fibre rod 22 may also be a right cylinder with a hexagonal base (see FIG. 7).

The fibre rod 22 may be produced from optical-quality glass, for example of the BK7 type.

A particular embodiment of the light guide 23 and scattering device 24 is described below.

In this particular embodiment, the light guide 23 and the scattering device 24 are formed by a set of optical fibres 25, for example made from polymethyl methacrylate (PMMA) making it possible to withstand heating temperatures of the scattering device ranging up to a maximum of approximately 80° C.

Each optical fibre 25 has a guide part 25-23 and a diffusing part 25-24. All the guide parts 25-23 of the optical fibres 25 then form together the light guide 23 and all the diffusing parts 25-24 of the optical fibres 25 form together the scattering device 24.

The optical fibres 23 enter the reaction chamber 11 through a cover 13 providing impermeability on each optical fibre 25.

The concentrated beam 5 enters through the free end of the guide part 25-23 of the optical fibres 25 and emerges diffuse along the diffusing part 25-24.

The diffusing part 25-24 of the optical fibres 25 is treated at least partially and advantageously entirely in order to confer diffusive properties thereto.

The diffusing part 25-24 of the optical fibres 25 may also be treated by surrounding it with an individual plastic sheath used to diffuse the light flux in the aqueous solution. In this way a layer of air is maintained between the individual plastic sheath and the rough patch of the diffusing part 25-24 of the optical fibres 25 caused by the laser treatment, which enables light flux diffusion in the liquid, when the material used for the optical fibres 25 does not permit it. The plastic sheath is treated at its ends so as to be sealed. At the cover 13 of the photobioreactor 1, the individual sheaths are moulded on by means of a resin to secure them and seal the cover 13.

The diffusing part 25-24 of the optical fibres 25 then has the property to diffuse the light radially over its entire length thus functioning as a of scattering device 24.

The advantage of using optical fibres 25 lies on the fact that they can be optimally distributed in the reaction chamber 11 of the photobioreactor 1, as described hereinafter. Furthermore, the use of individual plastic sheaths that is more rigid than the material of the optical fibres 25 allows for easier positioning of the diffusing parts 25-24 in the reaction chamber 11 when the optical fibres 25 are too flexible.

The guide parts 25-23 of the optical fibres 25 can be assembled into strands 26 (see FIG. 8), the diffusing parts 25-24 of the optical fibres 25 in the reaction chamber 11 being individualised.

Gratings 2g can be provided so as to be placed into a regular manner in the reaction chamber 11.

For example, when the photobioreactor 1 has a generally cylindrical shape with a disc-shaped cross-section and the length of which is positioned vertically, each grating 2g is advantageously formed by a circular frame 2g1 and parallel wires 2g2 tensioned inside the frame 2g1 and the ends of which are fixed to the circular frame 2g1. The frame 2g1 has more or less the dimensions of the cross-section of the reaction chamber 11, also cylindrical.

The frame 2g1, which here above is circular, may have another form matching the sides of the reaction chamber 11 of the photobioreactor in question.

These gratings 2g make it possible to obtain a triangular meshing of the diffusing parts 25-24 of the optical fibres 25, as will be explained hereinafter.

The module 2 may also comprise a near-infrared filter 27 for filtering the infrared and/or near infrared wavelengths of sunlight or the concentrated beam 5.

The filter 27 prevents the entrance of radiation in the near-infrared range (700 nm and beyond) which is unnecessary for the photosynthesis of the microorganisms (only visible light—between 400 and 700 nm—is useful) and which may cause an excessive temperature increase inside the reaction chamber 11 of the photobioreactor 1. The temperature increase inside the reaction chamber 11 beyond a certain threshold may kill the microorganisms. The temperature increase may also take place in the scattering device 24 and therefore damage it; thus the use of a filter 27 reduces accordingly the risk of damage to the scattering device 24.

Concentrator with Fresnel Lens

Module

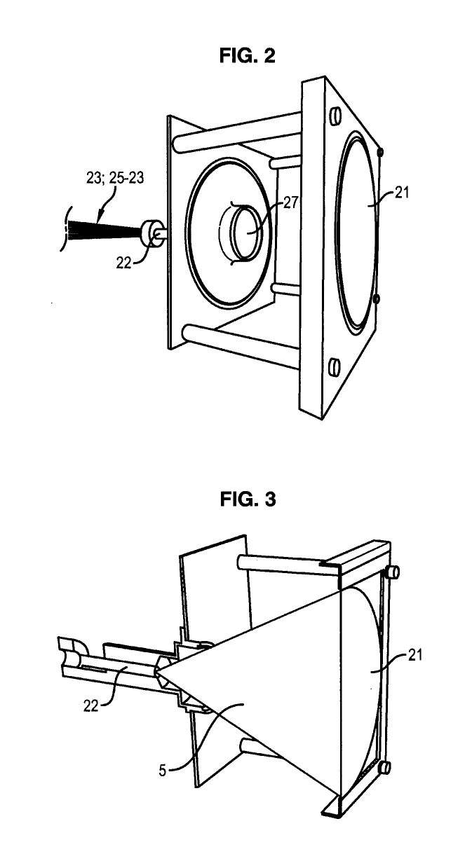

In a first particular embodiment of the module 2 described above and illustrated in FIGS. 2 and 3, the concentrator 1 may consist of a convergent lens. The convergent lens may be a Fresnel lens.

The use of a Fresnel lens makes it possible to obtain a concentrator 1 that is light and easy to install. The use of a Fresnel lens also reduces the manufacturing costs of the module 2. A Fresnel lens also enables concentration levels of several hundreds. The size of a Fresnel lens may be between several millimetres and several tens of centimetres.

A sun tracker 4 (see FIG. 12) is also provided for directing the rays of the sun to the Fresnel-lens concentrator 21. The sun tracker 4 is positioned facing the Fresnel-lens concentrator 21. The sun tracker 4 comprises a mirror 41 the inclination of which can be oriented by an inclination regulator 42 and the surface plane of which intersects horizontally the mid-plane of the Fresnel lens of the concentrator 21.

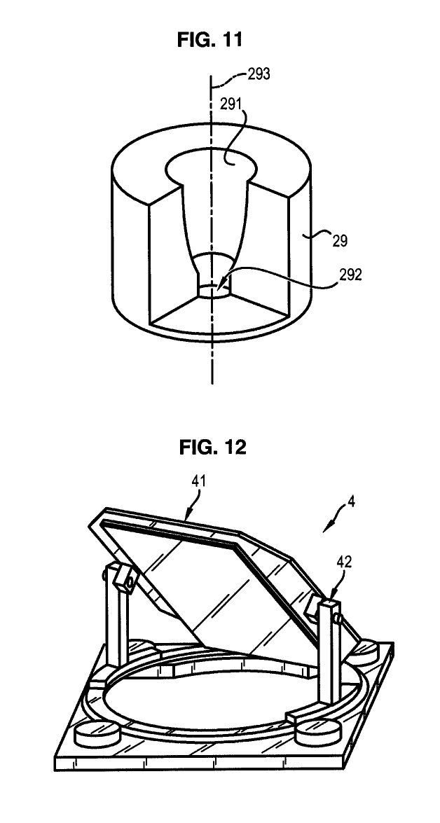

A compound-mirror collector 29 with an almost punctiform focus (or “compound parabolic concentrator”) illustrated in FIG. 11 can be provided for redirecting the rays coming from the Fresnel lens. The collector 29 is placed just in front of the focus of the Fresnel lens, between the Fresnel lens and the homogeniser 22.

The compound-mirror collector 29 with an almost punctiform focus has a symmetry of revolution and comprises an entry opening 291 and an exit opening 292 both centred on its axis of revolution 293. The geometric form of the collector 29 is constructed on the basis of an arc of a parabola and redirects any ray entering the entry opening 291 towards the exit opening 292 and therefore towards the homogeniser 22. The use of such a collector 29 allows superconcentration by a factor ranging from two to four. This collector 29 also compensates for low-precision tracking of the sun. The entry opening 291 of the collector 29 is sufficiently wide to ensure that the entire beam 25 concentrated by the Fresnel lens enters therein.

The filter 27 may be a film absorbing infrared/near-infrared wavelengths. The filter 27 may also be a hot mirror that has the property of reflecting the near-infrared wavelengths and being transparent to the visible light useful for photosynthesis.

The filter 27 may be positioned between the concentrator 21 and the collector 29. The filter 27 may also be positioned in front of the concentrator 21, which then receives only filtered light.

In the case of the Fresnel-lens concentrator 21, several modules 2 may be collected together in parallel and used for the same photobioreactor 1.

Panel for Capturing and Distributing Sunlight

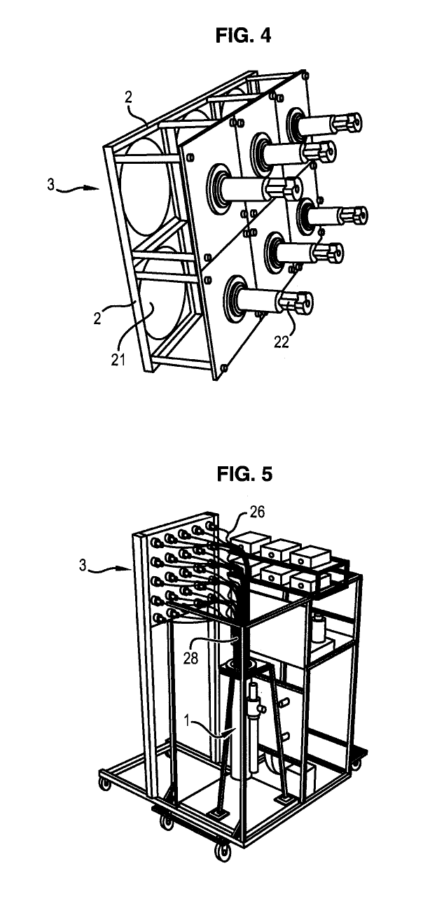

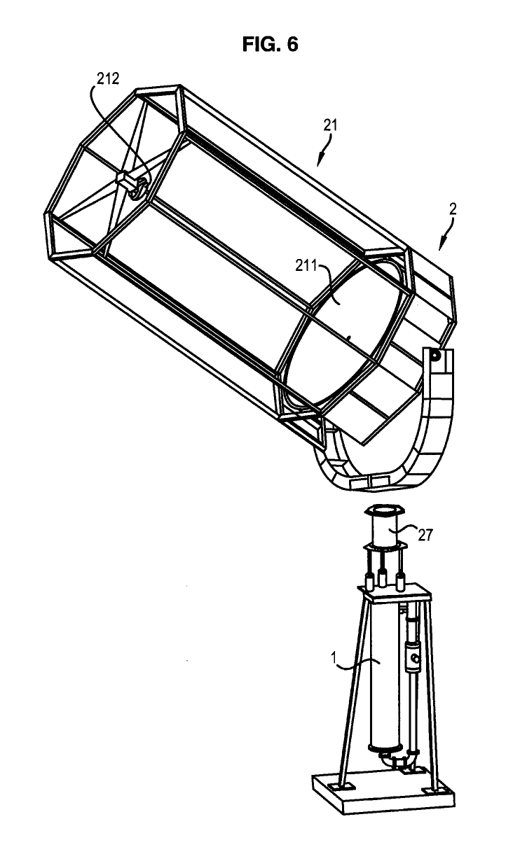

Several modules 2 as described above can be assembled in order to form a panel 3 for capturing and distributing sunlight, as shown in FIGS. 4 and 5.

In such a panel 3, the modules 2 are mounted in parallel.

When the light guide 23 and the scattering device 24 are formed by optical fibres 25 as described above, the guide parts 25-23 of the optical fibres 25 of a module are grouped together in strands 26.

The strands 26 of all the modules 2 are then grouped together at the cover 13 of the photobioreactor 1, the diffusing parts 25-24 of the optical fibres 25 of all the modules 2 being left individualised.

For example, twenty five (25) modules 2 are used for a photobioreactor 1. Nine hundred and seventy seven (977) optical fibres 25 are distributed in the reaction chamber 11 of the photobioreactor 1. The 977 optical fibres 25 are distributed in twenty five (25) strands 26 of thirty nine or forty (39 or 40) optical fibres 25, a module 2 comprising one strand 26.

In general terms, if N optical fibres 25 are to be distributed in the reaction chamber 11 of the photobioreactor 1, the number of modules 2 may vary from one to N with the distribution of the N fibres in strand(s).

Thus it is easy to modulate and adapt the size of the concentrator 21 to the requirements of the photobioreactor 1 and/or to the microorganisms.

In the case where gratings 2g are provided, there is not one set of gratings per module, but one set of gratings for the entire panel 3, that is to say at least three gratings 2g per panel 3.

### Cassegrain Concentrator

In a second particular embodiment illustrated in FIG. 6, the concentrator 21 can consist of a double-parabola system, known as “Cassegrain”, comprising a first parabola 211 and a second, smaller, parabola 212 positioned at the focus of the first parabola 211.

The first parabola 211 receives the light from the sun and reflects it towards the second smaller parabola 212, thus operating a first concentration. The second parabola 212 then reflects the light towards the homogeniser 22.

The first parabola 211 can be orientable when it is associated with a controlled sun-tracking system well known to persons skilled in the art. This affords optimum functioning at each moment in the day.

The second parabola 212 may also be orientable automatically so as to focus or defocus the concentrated beam 5 for control of the flux density sent to the homogeniser 22.

The filter 27 is arranged so as to receive the concentrated beam 5 reflected by the second parabola 212.

The filter 27 may be a liquid filter with a cooled double wall. Between the walls, an aqueous solution is added and makes it possible to filter both the undesirable ultraviolet radiation and the near-infrared radiation.

The filter 27 may also be a solid filter (for example a flat filter made from glass or other filter described above for the Fresnel-lens concentrator, only the size and shape being different) optionally cooled by water circulating in a double wall.

### Photobioreactor

One example of a photobioreactor 1 according to the invention comprises a module 2 as described above with a Fresnel or Cassegrain lens (in this case, only one module 2 is used) or at least two modules 2 as described above with a Fresnel lens (see FIGS. 5, 6 and 10).

The scattering device 24 is placed inside the reaction chamber 11 of the photobioreactor 1.

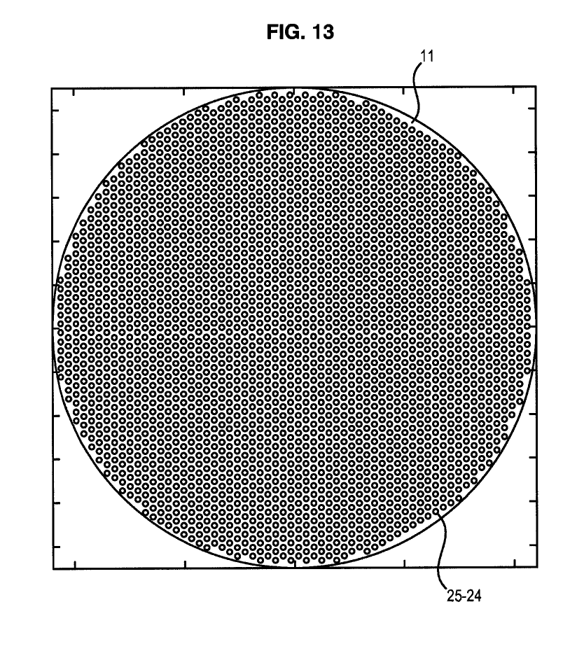

In the particular case where the light guide 23 and the scattering device 24 are formed by the same element composed of optical fibres 25 as described above, the diffusing parts 25-24 of the fibres 25 in the reaction chamber 11 may be tensioned parallel to one another and disposed in a triangular mesh (see FIG. 13).

The distance separating two optical fibres 25 in the reaction chamber 11 depends on the geometry of the photobioreactor 1, the expected biomass concentration (intensification) and the expected flux density inside. For example, for a typical flux density of 4-5 W/m² (in the range from 400 nm to 700 nm) and an aimed biomass concentration of 15 kg/m³, the optimum distance between the optical fibres is 2.3 mm, the optimisation of the whole leading then to an optimum diameter of the optical fibres 25 of 2.3 mm also, whatever the diameter of the reaction chamber 11. The total optimum volumetric occupation of the optical fibres 25 in the reaction chamber 11 is advantageously approximately 23% of the volume of the reaction chamber 11.

As the light flux of the concentrated beam 5 is homogeneously distributed by the homogeniser 22 into each of the optical fibres 25, the light flux density diffused by the diffusing parts 25-24 of the optical fibres 25 is homogeneous. In addition, as the surface of the optical fibres with lateral diffusion is much greater than the capture surface, the flux density at their surface is low. This makes it possible to function at the thermodynamic optimum of photosynthesis, and therefore to increase the surface productivities, but also to reduce the production of polysaccharides by the microorganisms. Thus the adhesion of these microorganisms, which is partly due to the production of polysaccharides, is minimised.

Still in this particular case, gratings 2g may be provided for positioning and holding the diffusing parts of the optical fibres 25 parallel and in a triangular mesh inside the reaction chamber 11.

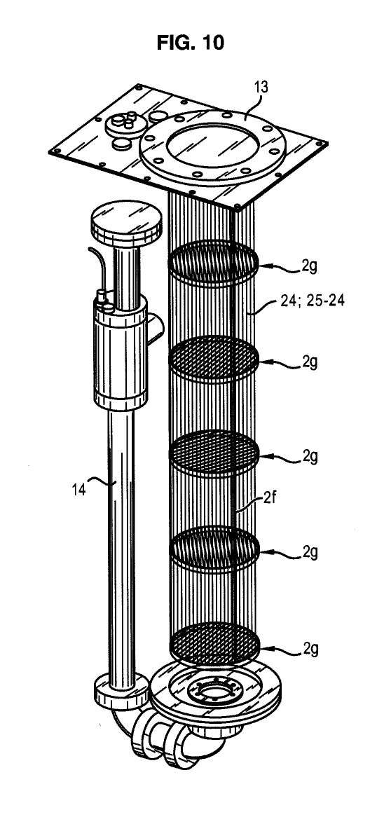

Thus, in order to obtain a triangular mesh, at least three gratings 2g as described above are arranged parallel one above the other in the reaction chamber 11. Two successive gratings 2g are rotated so that their respective wires 2g2 are tensioned in different directions and form an angle of ±60° as shown in FIG. 10. If the gratings 2g are counted from bottom to top and the direction of the wires of the first grating 2g is taken as reference, that is to say this direction marks the angle 0°, then the direction of the wires 2g2 of the second grating 2g marks the angle +60° or respectively −60°. Likewise the direction of the wires 2g2 of the third grating 2g situated above marks the angle +120° or respectively −120°. If a fourth grating 2g is used, the direction of its wires 2g2 marks the angle +180° or respectively −180°, which is equivalent to an angle of 0°.

The gratings 2g can be held at a distance one from another by cables 2f.

Thus the grilles 2g virtually form a triangular tubular mesh. In each of the tubular mesh openings the diffusing part 25-24 of an optical fibre 25 can be placed. The diffusing part 25-24 of each of the optical fibres 25 thus placed is held in place, thus ensuring light flux diffusion that is homogeneous inside the reaction chamber 11.

When individual sheaths for the diffusing parts 25-24 of the optical fibres are provided, these are moulded at the cover 13 by the resin whereas they are disposed according to the triangular mesh as described above and extend inside the reaction chamber 11 according to this triangular mesh. This is in particular used in the case of a panel 3 as described above, in which the strands 26 coming from several modules 2 are joined at the cover 13.

The individual sheaths therefore are used as support for the optical fibres 25 in the reaction chamber 11 in order to keep the optical fibres 25 in place according to the triangular mesh. In this case, the individual sheaths inside which the diffusing parts 25-24 of the optical fibres 25 are located pass through the gratings 2g, when these are provided, in the same way as the optical fibres 25 described above.

The reaction chamber 11 of the photobioreactor 1 may be of the double envelope type enabling the culture of microorganisms to be thermostatically controlled. For example, the walls of the cylindrical reaction chamber 11 are made from double-jacket stainless steel with a height to diameter ratio of approximately six.

If a near-infrared filter 27 is used, an excess of heat in the reaction chamber is avoided and it is no longer necessary to provide a heat-discharge means for the photobioreactor 1.

An original gas trap system 14 with an external recirculation loop and central return, duplicated by a metal porous part for the vertical injection of gas (air, CO₂, O₂) is provided for the photobioreactor 1 in order to ensure mixing of the liquid phase of the culture and gas-liquid mass transfers (CO₂/O₂ mainly) while allowing for a good circulation of fluids and good gas-liquid mass transfer in the reaction chamber with a minimum of pressure drops despite the presence of numerous optical fibres 25.

The metal porous part is placed at the base of the gas trap system 14 in an air box that surrounds it. Thus, the gases are brought by a radial feed and injected vertically through the metal porous part. The solution (liquid) is returned through the centre of the gas trap. The metal porous part affords better gas-light mass transfer by allowing for the formation of finer gas bubbles in the solution.

The effective circulation generated then minimises the adhesion of cells to the walls and/or to the scattering device.

1. A module for guiding sunlight for a photoreactor comprising a reaction chamber, the module comprising:
a concentrator placed outside the reaction chamber in order to concentrate the sunlight in a concentrated beam;
a scattering device to be placed inside the reaction chamber in order to introduce the concentrated beam into the reaction chamber and diffuse the concentrated beam in the reaction chamber;
wherein the module also comprises a homogeniser placed between the concentrator and the scattering device, outside the reaction chamber, in order to make the concentrated beam homogeneous before the concentrated beam enters the scattering device.

2. The module of claim 1, further comprising a light guide for guiding the light from the concentrator to the scattering device, the light guide being placed between the homogeniser and the scattering device outside the reaction chamber.

3. The module of claim 1, further comprising a near-infrared filter for filtering sunlight so that the light transmitted into the reaction chamber 11 is free from radiation in the near infrared.

4. The module of claim 1, wherein the concentrator is a Fresnel-lens concentrator comprising a Fresnel lens having a focus.

5. The module of claim 4, further comprising a compound-mirror collector with an almost punctiform focus placed between the Fresnel-lens concentrator and the homogeniser and in front of the focus of the Fresnel lens in order to redirect the concentrated beam.

6. The module of claim 1, wherein the concentrator is a concentrator of the Cassegrain type.

7. The module of claim 1, wherein the light guide and the scattering device form a single element composed of optical fibres;
wherein each optical fibre has a diffusing part, the diffusing parts of the optical fibres forming the scattering device; and
wherein each optical fibre has a guide part outside the reaction chamber, the guide parts of the optical fibres forming the light guide, the guide parts of the optical fibres being collected together into a strand.

8. A light-guide panel comprising at least two modules of claim 1, the modules being mounted in parallel.

9. A photoreactor comprising a reaction chamber, a module of claim 1 or a light-guide panel according to claim 8, the scattering device of the module or the diffusers of the panel being placed inside the reaction chamber.

10. A photoreactor comprising a reaction chamber, a module according to claim 7, wherein the diffusing parts of the optical fibres of the module or of the panel being arranged tensioned and parallel to each other in the reaction chamber into a triangular mesh.

11. The photoreactor of claim 10, further comprising at least three gratings placed parallel one above another, each being formed by a frame and tensioned wires in the frame parallel to one another in one direction, the direction of the wires of a gratings forming with the direction of the wires of another gratings above an angle of ±60° to hold the diffusing parts of the optical fibres in a triangular mesh.

12. The photoreactor of one of claim 10 or 11, further comprising a gas trap with an external recirculation loop and a central return for mixing the content of the reaction chamber effectively.
